(19) United States
(12) Patent Application Publication
(10) Pub. No.: US 2007/0287703 A1
(43) Pub. Date: Dec. 13, 2007

(54) Fused Pyrimidones Useful in the Treatment and the Prevention of Cancer

(75) Inventors: Michael Howard Block, Sudbury, MA (US); Audrey Davies, Waltham, MA (US); Daniel John Russell, Natick, MA (US); Marie-Elena Theoclitou, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 11/572,445

(22) PCT Filed: Jul. 21, 2005
(86) PCT No.: PCT/GB05/02845

§ 371 Date: Jan. 22, 2007

Related U.S. Application Data
(60) Provisional application No. 60/590,246, filed on Jul. 22, 2004.

(51) Int. Cl. A61K 31/517; A61K 31/54; A61P 35/04; C07D 239/70; C07D 417/04

(52) U.S. Cl. 514/226.8; 514/258.1; 544/253; 544/53; 544/55

(57) ABSTRACT

Compounds which possess Eg5 inhibitory activity and are useful for their anti-cell-proliferation (such as anti-cancer) activity and thus in methods of treatment of the human or animal body are described.

The present invention relates to chemical compounds, processes for preparing them, their pharmaceutical compositions and methods of use. In addition, the present invention relates to therapeutic methods for the treatment and prevention of cancers and to the use of these chemical compounds in the manufacture of a medicament for use in the treatment and prevention of cancers.

One sub-class of anti-cancer drugs (taxanes, vinca-alkaloids) now used extensively in the clinic is directed at microtubules and blocks the cell division cycle by interfering with normal assembly or disassembly of the mitotic spindle (see Chabner, B. A., Ryan, D. P., Paz-Ares, 1., Garcia-Carbonero, R., and Calabresi, P: Antineoplastic agents. In Hardman, J. G., Limbird, L. E., and Gilman, A. G., eds. Goodman and Gilman's The Pharmacological Basis of Therapeutics, 10^{th} edition, 2001, The MacGraw-Hill Companies, Inc). Taxol® (paclitaxel), one of the most effective drugs of this class, is a microtubule stabilizer. It interferes with the normal growth and shrinkage of microtubules thus blocking cells in the metaphase of mitosis. Mitotic block is often followed by slippage into the next cell cycle without having properly divided, and eventually by apoptosis of these abnormal cells (Blagosklonny, M. V. and Fojo, T.: Molecular effects of paclitaxel: myths and reality (a critical review). Int J Cancer 1999, 83:151-156.)

Some of the side effects of treatment with paclitaxel are neutropenia and peripheral neuropathy. Paclitaxel is known to cause abnormal bundling of microtubules in interphase cells. In addition, some tumor types are refractory to treatment with paclitaxel, and other tumors become insensitive during treatment. Paclitaxel is also a substrate for the multi-drug resistance pump, P-glycoprotein ((see Chabner et al., 2001).

Thus, there is a need for effective anti-mitotic agents that have fewer side effects than anti-microtubule drugs, and also for agents that are effective against taxane-resistant tumors.

Kinesins are a large family of molecular motor proteins, which use the energy of Adenosine 5′-triphosphate (ATP) hydrolysis to move in a stepwise manner along microtubules. For a review, see Sablin, E. P.: Kinesins and microtubules: their structures and motor mechanisms. Curr Opin Cell Biol 2000, 12:35-41 and Schief, W. R. and Howard, J.: Conformational changes during kinesin motility. Curr Opin Cell Biol 2001, 13:19-28.

Some members of this family transport molecular cargo along microtubules to the sites in the cell where they are needed. For example, some kinesins bind to vescicles and transport them along microtubules in axons. Several family members are mitotic kinesins, as they play roles in the reorganization of microtubules that establishes a bipolar mitotic spindle. The minus ends of the microtubules originate at the centrosomes, or spindle poles, whilst the plus ends bind to the kinetochore at the centromeric region of each chromosome. The mitotic spindle lines up the chromosomes at metaphase of mitosis and coordinates their movement apart and into individual daughter cells at anaphase and telophase (cytokinesis). See Alberts, B., Bray, D., Lewis, J., Raff, M., Roberts, K., and Watson, J. D., Molecular Biology of the Cell, 3^{rd} edition, Chapter 18, The Mechanics of Cell Division, 1994, Garland Publishing, Inc. New York.

HsEg5 (homo sapiens Eg5) (Accession X85137; see Blangy, A., Lane H. A., d'Heron, P., Harper, M., Kress, M. and Nigg, E. A.: Phosphorylation by p34cdc2 regulates spindle association of human Eg5, a kinesin-related motor essential for bipolar spindle formation in vivo. Cell 1995, 83(7): 1159-1169) or, KSP (kinesin spindle protein), is a mitotic kinesin whose homologs in many organisms have been shown to be required for centrosome separation in the prophase of mitosis, and for the assembly of a bipolar mitotic spindle. For a review see Kashina, A. S., Rogers, G. C., and Scholey, J. M.: The bimC family of kinesins: essential bipolar mitotic motors driving centrosome separation. Biochem Biophys Acta 1997, 1357: 257-271. Eg5 forms a tetrameric motor, and it is thought to cross-link microtubules and participate in their bundling (Walczak, C. E., Vemos, I., Mitchison, T. J., Karsenti, E., and Heald, R.: A model for the proposed roles of different microtubule-based motor proteins in establishing spindle bipolarity. Curr Biol 1998, 8:903-913). Several reports have indicated that inhibition of Eg5 function leads to metaphase block in which cells display monastral spindles. Recently an Eg5 inhibitor called monastrol was isolated in a cell-based screen for mitotic blockers (Mayer, T. U., Kapoor, T. M., Haggarty, S. J., King, R. w., Schreiber, S. L., and Mitchison, T. J.: Small molecule inhibitor of mitotic spindle bipolarity identified in a phenotype-based screen. Science 1999, 286: 971-974).

Monastrol treatment was shown to be specific for Eg5 over kinesin heavy chain, another closely related motor with different functions (Mayer et al., 1999). Monastrol blocks the release of ADP (adenosine 5′-diphosphate) from the Eg5 motor (Maliga, Z., Kapoor, T. M., and Mitchison, T. J.: Evidence that monastrol is an allosteric inhibitor of the mitotic kinesin Eg5. Chem & Biol 2002, 9: 989-996 and DeBonis, S., Simorre, J.-P., Crevel, I., Lebeau, L, Skoufias, D. A., Blangy, A., Ebel, C., Gans, P., Cross, R., Hackney, D. D., Wade, R. H., and Kozielski, F.: Interaction of the mitotic inhibitor monastrol with human kinesin Eg5. Biochemistry 2003, 42: 338-349) an important step in the catalytic cycle of kinesin motor proteins (for review, see Sablin, 2000; Schief and Howard, 2001). Treatment with monastrol was shown to be reversible and to activate the mitotic spindle checkpoint which stops the progress of the cell division cycle until all the DNA is in place for appropriate division to occur (Kapoor, T. M., Mayer, T. U., Coughlin, M. L., and Mitchison, T. J.: Probing spindle assembly mechanisms with monastrol, a small molecule inhibitor of the mitotic kinesin, Eg5. J Cell Biol 2000, 150(5): 975-988). Recent reports also indicate that inhibitors of Eg5 lead to apoptosis of treated cells and are effective against several tumor cell lines and tumor models (Mayer et al., 1999).

Although Eg5 is thought to be necessary for mitosis in all cells, one report indicates that it is over-expressed in tumor cells (International Patent Application WO 01/31335), suggesting that they may be particularly sensitive to its inhibition. Eg5 is not present on the microtubules of interphase cells, and is targeted to microtubules by phosphorylation at an early point in mitosis (Blangy et al., 1995. See also; Sawin, K. E. and Mitchison, T. J.: Mutations in the kinesin-like protein Eg5 disrupting localization to the mitotic spindle. Proc Natl Acad Sci USA 1995, 92(10): 4289-4293), thus monastrol has no detectable effect on microtubule arrays in interphase cells (Mayer et al., 1999). Another report suggests that Eg5 is involved in neuronal development in the mouse, but it disappears from neurons soon after birth, and thus Eg5 inhibition may not produce the peripheral neuropathy associated with treatment with paclitaxel and other anti-microtubule drugs (Ferhat, L., Expression of the mitotic motor protein Eg5 in postmitotic neurons: implications for neuronal development. J Neurosci 1998, 18(19): 7822-7835). Herein we describe the isolation of a class of specific and potent inhibitors of Eg5, expected to be useful in the treatment of neoplastic disease.

Certain pyrimidones have recently been described as being inhibitors of KSP (WO 03/094839, WO 03/050122, WO 03/050064, WO 03/049679, WO 03/049527, WO 04/078758, WO 04/106492, WO 04/111058 and WO 03/099211).

In accordance with the present invention, the present inventors have discovered novel chemical compounds which possess Eg5 inhibitory activity and are accordingly useful for their anti-cell-proliferation (such as anti-cancer) activity and are therefore useful in methods of treatment of the human or animal body.

Therefore in accordance with the present invention there is provided a compound of formula (I): 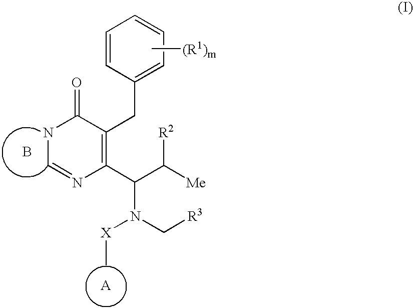
wherein:

R¹ is fluoro;

m is 0-5;

R² is hydrogen or methyl;

R³ is a carbon linked —NR⁴— containing heterocyclic ring or R³ is C₁₋₃alkyl substituted by —NR⁵R⁶; wherein R³ may be optionally substituted on carbon by one or more R⁷;

X is —C(O)— or —CH₂—;

Ring A is a carbocyclyl or heterocyclyl; wherein Ring A may be optionally substituted on carbon by one or more R⁸; and wherein if said heterocyclyl contains an additional NH that nitrogen may be optionally substituted by R⁹;

Ring B is fused to the pyrimidone ring of formula (I) as shown and is a 5 or 6 membered fused carbocyclic ring or 5 or 6 membered fused heterocyclic ring; wherein Ring B may be optionally substituted on carbon by one or more R¹⁰; and wherein if said 5 or 6 membered fused heterocyclic ring contains an additional NH that nitrogen may be optionally substituted by R¹¹;

R⁴ is selected from hydrogen, C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R⁵ and R⁶ are independently hydrogen or C₁₋₆alkyl; or R⁵ and R⁶ together with the nitrogen to which they are attached form a nitrogen containing heterocycle; wherein said C₁₋₆alkyl or said nitrogen containing heterocycle may be independently optionally substituted on carbon by one or more R¹²; and wherein if said nitrogen containing heterocycle contains an additional NH that nitrogen may be optionally substituted by R¹³;

R⁸, R¹⁰ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino; wherein R⁸, R¹⁰ and R¹² may be independently optionally substituted by R¹⁴;

R⁹, R¹¹ and R¹³ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R⁷ and R¹⁴ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In this specification the term “alkyl” includes both straight and branched chain alkyl groups. For example, “C₁₋₆alkyl” includes C₁₋₄alkyl, C₁₋₃alkyl, methyl, ethyl, propyl, isopropyl and t-butyl. However, references to individual allyl groups such as ‘propyl’ are specific for the straight chained version only and references to individual branched chain alkyl groups such as ‘isopropyl’ are specific for the branched chain version only. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “carbon linked —NR⁴— containing heterocyclic ring” is a saturated, partially saturated or unsaturated, mono or bicyclic ring containing 3-12 atoms of which at least one atom is chosen from the nitrogen which is substituted by R⁴ and wherein the other atoms are selected from carbon and optionally 1-3 additional heteroatoms selected from nitrogen, sulphur or oxygen atoms, wherein a —CH₂— group can optionally be replaced by a —C(O)— and a ring nitrogen atom or a ring sulphur atom may be optionally oxidised to form the N— and/or S-oxide(s). Suitable examples of a “carbon linked —NR⁴— containing heterocyclic ring” include piperidinyl, piperazinyl, morpholinyl, aziridinyl, azetidinyl, indolyl, pyrazolinyl and imidazolyl.

A “5 or 6 membered fused carbocyclic ring” fused to the pyrimidone ring of formula (I) is a saturated, partially saturated or unsaturated, monocyclic ring containing 5 or 6 carbon atoms, wherein a —CH₂— group can optionally be replaced by a —C(O)—. Examples of “5 or 6 membered fused carbocyclic ring” fused to the pyrimidone ring of formula (I) are 6,7,8,9-tetrahydro-4H-pyrido[1,2-a]pyrimidin-4-one, 4H-pyrido[1,2-a]pyrimidin-4-one. pyrrolo[1,2-a]pyrimidin-4(6H)-one and 7,8-dihydropyrrolo[1,2-α]pyrimidin-4(6H)-one. For the avoidance of doubt, it is to be understood that, once fused to the pyrimidone of formula (I), the “5 or 6 membered fused carbocyclic ring” will contain the nitrogen of the pyrimidinone ring.

A “5 or 6 membered fused heterocyclic ring” fused to the pyrimidone ring of formula (I) is a saturated, partially saturated or unsaturated, monocyclic ring containing 5 or 6 atoms of which at least one atom is chosen from nitrogen, sulphur or oxygen, wherein a —CH₂— group can optionally be replaced by a —C(O)— and a ring nitrogen atom or a ring sulphur atom may be optionally oxidised to form the N— and/or S-oxide(s). Examples of a “5 or 6 membered fused heterocyclic ring” fused to the pyrimidone ring of formula (I) are 2,3-dihydro-5-oxo-5H-[1,3]thiazolo[3,2-a]pyrimidine, 3,4-dihydro-6-oxo-2H,6H-pyrimido[2,1-b][1,3]thiazine and 5-oxo-5H-[1,3]thiazolo[3,2-a]pyrimidine.

A “carbocyclyl” is a saturated, partially saturated or unsaturated, mono or bicyclic ring containing 4-12 carbon atoms, wherein a —CH₂— group can optionally be replaced by a —C(O)—. Particularly a “carbocyclyl” is a saturated, partially saturated or unsaturated, monocyclic ring containing 5 or 6 atoms or a bicyclic ring containing 9 or 10 atoms wherein a —CH₂— group can optionally be replaced by a —C(O)—. Examples and suitable values of the term “carbocyclyl” are cyclopropyl, cyclohexyl, phenyl and naphthyl.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring nitrogen atom or a ring sulphur atom may be optionally oxidised to form the N— and/or S-oxide(s). Particularly a “heterocyclyl” is a saturated, partially saturated or unsaturated, monocyclic ring containing 5 or 6 atoms or a bicyclic ring containing 9 or 10 atoms of which at least one atom is chosen from nitrogen, sulphur or oxygen, which may, unless otherwise specified, be carbon or nitrogen linked, a —CH₂— group can optionally be replaced by a —C(O)—and a ring sulphur atom may be optionally oxidised to form the S-oxide(s). Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, 4-pyridone, 1-isoquinolone, 2-pyrrolidone and 4-thiazolidone.

Where “R⁵ and R⁶ together with the nitrogen to which they are attached form a nitrogen containing heterocycle” said “nitrogen containing heterocycle” is a saturated, partially saturated or fully unsaturated, mono or bicyclic ring containing 4-12 atoms, one atom of which is the nitrogen atom to which R⁵ and R⁶ are attached to, and the other atoms are either all carbon atoms or they are carbon atoms and 1-3 heteroatoms chosen from nitrogen, sulphur or oxygen, wherein a —CH₂— group can optionally be replaced by a —C(O)—, and a ring nitrogen atom or a ring sulphur atom may be optionally oxidised to form the N— and/or S-oxide(s). It will be appreciated that where “R⁵ and R⁶ together with the nitrogen to which they are attached form a nitrogen containing heterocycle” this nitrogen atom is not quatemised, i.e. a neutral compound is formed. Examples and suitable values of the term “nitrogen containing heterocycle” are azetidinyl, morpholino, piperidyl, piperazinyl, pyrrolidinyl, thiomorpholino, pyrrolinyl, homopiperazinyl, pyrrolyl, pyrazolyl, pyrazolinyl, imidazolyl, imidazolinyl, imidazolidinyl, pyrazolidinyl and triazolyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “C₁₋₆alkylsulphonyl” include mesyl, ethylsulphonyl and t-butylsulphonyl. Examples of “C₁₋₆alkylsulphonylamino” include mesylamino, ethylsulphonylamino and t-butylsulphonyl. Examples of “C₁₋₃alkylene” are methylene, ethylene and propylene.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product. The pharmaceutically acceptable salts of the invention also include salts prepared with one of the following acids benzene sulfonic acid, fumaric acid, methanesulfonic acid, naphthalene-1,5-disulfonic acid, naphthalene-2-sulfonic acid or L-tartaric acid.

Thus in one aspect of the invention there is provided a compound of the invention, particularly one of the Examples described herein, as a pharmaceutically acceptable salt, particularly a benzene sulfonic acid, famaric acid, methanesulfonic acid, naphthalene-1,5-disulfonic acid, naphthalene-2-sulfonic acid or L-tartaric acid salt.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin. Alternatively, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

A variety of compounds in the present invention may exist in particular geometric or stereoisomeric forms. The present invention takes into account all such compounds, including cis- and trans isomers, R— and S— enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

Particular values of variable groups are as follows. Such values may be used where appropriate with any of the definitions, claims or embodiments defined hereinbefore or hereinafter.

m is 0.

R² is hydrogen.

R² is methyl.

R³ is a carbon linked —NR⁴— containing heterocyclic ring; wherein R³ may be optionally substituted on carbon by one or more R⁷.

R³ is C₁₋₃allyl substituted by —NR⁵R⁶; wherein R³ may be optionally substituted on carbon by one or more R⁷.

R³ is C₁₋₃alkyl substituted by —NR⁵R⁶.

R³ is methyl or ethyl substituted by —NR⁵R⁶; wherein R⁵ and R⁶ are independently hydrogen or methyl.

R³ is methyl or ethyl substituted by —NR⁵R⁶; wherein both R⁵ and R⁶ are hydrogen or both R⁵ and R¹ are methyl.

R⁵ and R⁶ are independently hydrogen or C₁₋₆alkyl.

R⁵ and R⁶ are independently hydrogen or methyl.

Both R⁵ and R⁶ are hydrogen or both R⁵ and R⁶ are methyl.

X is —C(O)—.

X is —CH₂—.

Ring A is a carbocyclyl; wherein Ring A may be optionally substituted on carbon by one r more R⁸.

Ring A is phenyl or naphthyl; wherein Ring A may be optionally substituted on carbon by one or more R⁸.

Ring A is a heterocyclyl; wherein Ring A may be optionally substituted on carbon by one or more R⁸; and wherein if said heterocyclyl contains an additional NH that nitrogen may be optionally substituted by R⁹.

Ring A is a carbocyclyl; wherein Ring A may be optionally substituted on carbon by one or more R⁸; wherein R⁸ is halo, C₁₋₆alkyl or C₁₋₆alkoxy.

Ring A is phenyl, naphthyl or benzothienyl; wherein Ring A may be optionally substituted on carbon by one or more R⁸; wherein R⁸ is fluoro, chloro, bromo, methyl or methoxy.

Ring A is phenyl or naphthyl; wherein Ring A may be optionally substituted on carbon by one or more R⁸; wherein R⁸ is fluoro, chloro, methyl or methoxy.

Ring A is 4-methyphenyl, 4-methoxyphenyl, 3-fluoro-4-methylphenyl, naphth-2-yl, 4-chlorophenyl, 2,3 dichlorophenyl or 4-bromophenyl.

Ring A is 4-methyphenyl, 4-methoxyphenyl, 3-fluoro-4-methylphenyl, naphth-2-yl or 4-chlorophenyl.

Ring B is a 5 or 6 membered fused carbocyclic ring; wherein Ring B may be optionally substituted on carbon by one or more R¹⁰.

Ring B is a 5 or 6 membered fused heterocyclic ring; wherein Ring B may be optionally substituted on carbon by one or more R¹⁰; and wherein if said 5 or 6 membered fused heterocyclic ring contains an additional NH that nitrogen may be optionally substituted by R¹¹.

Ring B is a 5 or 6 membered fused carbocyclic ring or a 5 or 6 membered fused heterocyclic ring.

Ring B and the pyrimidone to which it is fused forms 2,3-dihydro-5-oxo-5H-[1,3]thiazolo[3,2-a]pyrimidine, 3,4-dihydro-6-oxo-2H,6H-pyrimido[2,1-b][1,3]thiazine, 5-oxo-5H-[1,3]thiazolo[3,2-a]pyrimidine or 4-oxo-6,7,8,9-tetrahydro-4H-pyrido[1,2-a]pyrimidin-2-yl.

Therefore in a further aspect of the invention, there is provided a compound of formula (I) as depicted above wherein:

m is 0;

R² is hydrogen;

R² is methyl;

R³ is C₁₋₃alkyl substituted by —NR⁵R⁶;

X is —C(O)— or —CH₂—;

Ring A is a carbocyclyl; wherein Ring A may be optionally substituted on carbon by one or more R⁸;

Ring B is a 5 or 6 membered fused carbocyclic ring or a 5 or 6 membered fused heterocyclic ring;

R⁵ and R⁶ are independently hydrogen or C₁₋₆alkyl;

R⁸ is halo, C₁₋₆alkyl or C₁₋₆alkoxy;

or a pharmaceutically acceptable salt thereof.

Therefore in a further aspect of the invention, there is provided a compound of formula (I) as depicted above wherein:

m is 0;

R² is hydrogen;

R³ is methyl or ethyl substituted by —NR⁵R⁶;

X is —C(O)— or —CH₂—;

Ring A is 4-methyphenyl, 4-methoxyphenyl, 3-fluoro-4-methylphenyl, naphth-2-yl or 4-chlorophenyl;

Ring B and the pyrimidone to which it is fused forms 2,3-dihydro-5-oxo-5H-[1,3]thiazolo[3,2-a]pyrimidine, 3,4-dihydro-6-oxo-2H,6H-pyrimido[2,1-b][1,3]thiazine, 5-oxo-5H-[1,3]thiazolo[3,2-a]pyrimidine or 4-oxo-6,7,8,9-tetrahydro-4H-pyrido[1,2-a]pyrimidin-2-yl;

R⁵ and R⁶ are independently hydrogen or methyl;

or a pharmaceutically acceptable salt thereof.

In another aspect of the invention, preferred compounds of the invention are any one of the Examples or a pharmaceutically acceptable salt or thereof.

Another aspect of the present invention provides a process for preparing a compound of formula (I) or a pharmaceutically acceptable salt thereof which process (wherein variable groups are, unless otherwise specified, as defined in formula (I)) comprises of:
Process a) when X is —C(O)—; reacting a quinazolinone of the formula (II) 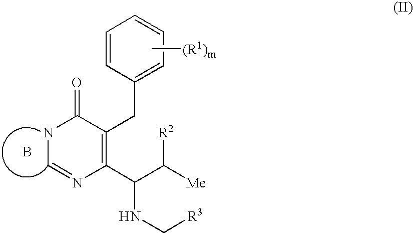
with an acid of formula (III): 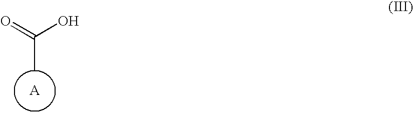
or an activated acid derivative thereof;
Process b) where X is —CH₂—; reacting a compound of the formula (II) with a compound of formula (V): 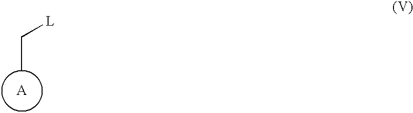
wherein L is a displaceable group;
Process c) for compounds of formula (I) wherein R³ is C₁₋₃alkyl substituted by —NR⁵R⁶ and optionally substituted on carbon by one or more R⁷; reacting a compound of formula (VI): 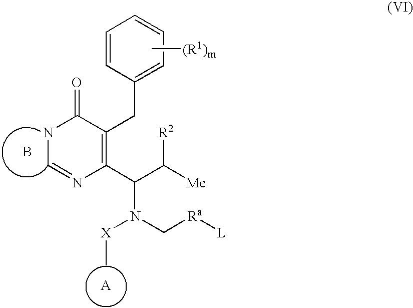
wherein R^{a} is C₁₋₃alkylene optionally substituted on carbon by one or more R⁷; and wherein L is a displaceable group; with an compound of formula (VII):
HNR⁵R⁶ (VII)
Process d) reaction of an amine of formula (VIII): 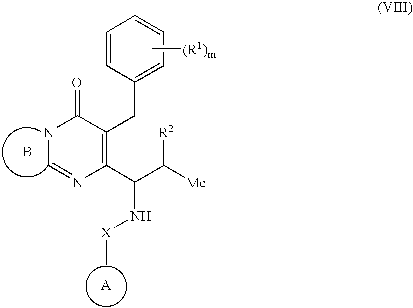
with a compound of formula (IX): 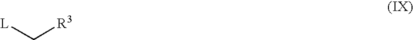
wherein L is a displaceable group; and thereafter if necessary:
i) converting a compound of the formula (I) into another compound of the formula (I);
ii) removing any protecting groups;
iii) forming a pharmaceutically acceptable salt.

L is a displaceable group, suitable values for L are for example, a halo for example a chloro or bromo.

Specific reaction conditions for the above reactions are as follows.

Process a) Amines of formula (II) and acids of formula (III) may be coupled together in the presence of a suitable coupling reagent. Standard peptide coupling reagents known in the art can be employed as suitable coupling reagents, or for Example carbonyldiimidazole and dicyclohexyl-carbodiimide, optionally in the presence of a catalyst such as dimethylaminopyridine or 4-pyrrolidinopyridine, optionally in the presence of a base for Example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for Example acid chlorides, and active esters, for Example pentafluorophenyl esters. The reaction of these types of compounds with amines is well known in the art, for Example they may be reacted in the presence of a base, such as those described above, and in a suitable solvent, such as those described above. The reaction may conveniently be performed at a temperature in the range of −40 to 40° C.

Amines of formula (II) may be prepared according to Scheme 1:

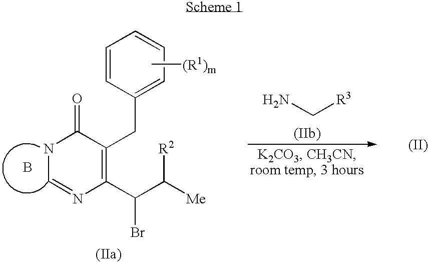

Compounds of formula (IIa), (IIb) and (III) are commercially available compounds, or they are known in the literature, or they are prepared by standard processes known in the art. Process b) Compounds of formula (II) and (V) can be reacted in an appropriate solvent with a base such as potassium carbonate. For example, compounds of formula (II) and (V) can be heated in dimethyl formamide or acetonitrile as solvent and potassium carbonate to yield compounds of formula (I).

Compounds of formula (V) are commercially available compounds, or they are known in the literature, or they are prepared by standard processes known in the art.

Process c) Compounds of formula (VI) and (VII) can be reacted together in an appropriate solvent in the presence of base. For example, compounds of formula (VI) and (VII) can be reacted in the presence of potassium carbonate in dimethylformamide at an elevated temperature.

Amines of formula (VI) may be prepared according to Scheme 2:

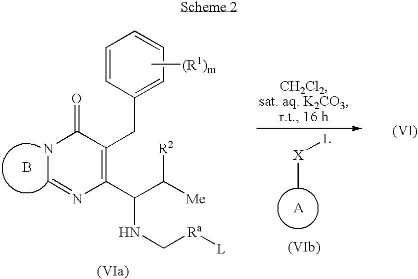

Compounds of formula (VII) are commercially available compounds, or they are known in the literature, or they are prepared by standard processes known in the art.

Process d) Compounds of formula (VIII) and (IX) can be reacted in an appropriate solvent with a base such as lithium t-butoxide. For example, compounds of formula (II) and (V) can be reacted in tetrahydrofuran as solvent and lithium t-butoxide at −40° C. to yield compounds of formula (I).

Amides of formula (VIII) may be prepared according to Scheme 3:

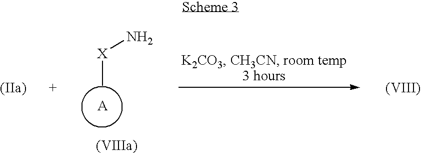

Compounds of formula (VIIIa) and (IX) are commercially available compounds, or they are known in the literature, or they are prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, allylation of substituents and oxidation of substituents. The reagents and reaction conditions for such procedures are well known in the chemical art.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate).

A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Assay

Malachite Green Assay

Enzymatic activity of the Eg5 motor and effects of inhibitors was measured using a malachite green assay, which measures phosphate liberated from ATP, and has been used previously to measure the activity of kinesin motors (Hackney D D, and Jiang W, Assays for kinesin microtubule-stimulated ATPase activity; Methods in Molecular Biology vol 164:65-71 (2001)). Enzyme was recombinant HsEg5 motor domain (amino acids 1-369-8His) and was added at a final concentration of 6 nM to 100 μl reactions. Buffer consisted of 25 mM PIPES(piperazine-1,4-bis(2-ethane-sulfonic acid)/KOH, pH 6.8, 2 mM MgCl₂, 1 mM EGTA(ethylene glycol-bis(2-aminoethyl ether)-N,N,N′N′-tetraacetic acid), 1 mM DTT (dithiotheitol), 0.01% Triton X-100 and 5 μM paclitaxel. Malachite green/ammonium molybdate reagent was prepared as follows: for 800 ml final volume, 0.27 g of Malachite Green (J. T. Baker) was dissolved in 600 ml of H₂O in a polypropylene bottle. 8.4 g Ammonium molybdate (Sigma) was dissolved in 200 ml 4N HCl. The solutions were mixed for 20 min and filtered through 0.02 μm filter directly into a polypropylene container.

5 μl of compound diluted in 12% DMSO was added to the wells of 96 well plates. 80 μl of enzyme diluted in buffer solution above was added per well and incubated with compound for 20 min. After this pre-incubation, substrate solution containing 2 mM ATP (adenosine 5′-tri-phosphate)(final concentration: 300 μM) and 6.053 μM polymerized tubulin (final concentration: 908 nM) in 15 μl of buffer were then added to each well to start reaction. Reaction was mixed and incubated for an additional 20 min at room temperature. The reactions were then quenched by the addition of 150 μl malachite green/ammonium molybdate reagent, and absorbance read at 650 nanometers exactly 5 min after quench using a Spectramax Plus plate reader (Molecular Devices). Data was graphed and IC₅₀s calculated using ExCel Fit (Microsoft).

Compounds of the present invention recorded an IC₅₀ in the range of 1 nM-9 μM in the above assay. Specifically the following results were obtained.

| Example | IC₅₀ |
|---|---|
| 4 | 331 nM |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their Eg5 inhibitory properties. Accordingly the compounds of the present invention are expected to be useful in the treatment of diseases or medical conditions mediated alone or in part by Eg5, i.e. the compounds may be used to produce a Eg5 inhibitory effect in a warm-blooded animal in need of such treatment.

Thus the compounds of the present invention provide a method for treating cancer characterised by inhibition of Eg5, i.e. the compounds may be used to produce an anti-cancer effect mediated alone or in part by the inhibition of Eg5.

The compounds of the present invention have utility for the treatment of neoplastic disease by inhibiting the microtubule motor protein HsEg5. Methods of treatment target Eg5 activity, which is required for the formation of a mitotic spindle and therefore for cell division. Thus, inhibitors of Eg5 have been shown to block cells in the metaphase of mitosis leading to apoptosis of effected cells, and to therefore have anti-proliferative effects. Thus Eg5 inhibitors act as modulators of cell division and are expected to be active against neoplastic disease such as carcinomas of the breast, ovary, lung, colon, prostate or other tissues, as well as multiple myeloma leukemias, for example myeloid leukemia, acute lymphoblastic leukemia, chronic myeloid leukemia, chronic lymphocytic leukemia, and lymphomas for example Hodgkins disease and non-Hodgkins lymphoma, tumors of the central and peripheral nervous system, and other tumor types such as melanoma, fibrosarcoma, Ewing's sarcoma and osteosarcoma. Eg5 inhibitors are also expected to be useful for the treatment other proliferative diseases including but not limited to autoimmune, inflammatory, neurological, and cardiovascular diseases.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a Eg5 inhibitory effect in a warm-blooded animal such as man.

According to a further aspect of the invention there is provided the use of a compound of the formula (I), or a pharmaceutically acceptable salt thereof, as defined hereinbefore in the manufacture of a medicament for use in the production of an anti-proliferative effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided a compound of the formula (I), or a pharmaceutically acceptable salt thereof, as defined herein before in the manufacture of a medicament for use in the treatment of carcinomas of the breast, ovary, lung, colon or prostate, leukemias and lymphomas, tumors of the central and peripheral nervous system, melanoma, fibrosarcoma and osteosarcoma.

According to a further feature of the invention, there is provided the use of a compound of the formula (I), or a pharmaceutically acceptable salt thereof, as defined herein before in the manufacture of a medicament for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

According to a further feature of this aspect of the invention there is provided a method for producing a Eg5 inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method of producing an anti-proliferative effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating carcinomas of the breast, ovary, lung, colon or prostate, leukemias and lymphomas, tumors of the central and peripheral nervous system, melanoma, fibrosarcoma and osteosarcoma, in a warm-blooded animal, such as man, in need of such treatment which comprises administering to said animal an effective amount of a compound of formula (I) or a pharmaceutically acceptable salt thereof as defined herein before.

According to an additional feature of this aspect of the invention there is provided a method of treating carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a Eg5 inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-proliferative effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of carcinomas of the breast, ovary, lung, colon or prostate, leukemias and lymphomas, tumors of the central and peripheral nervous system, melanoma, fibrosarcoma and osteosarcoma in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

According to a further aspect of the invention there is provided the use of a compound of the formula (I), or a pharmaceutically acceptable salt thereof, as defined hereinbefore in the production of an Eg5 inhibitory effect in a warm-blooded animal such as man.

According to a further aspect of the invention there is provided the use of a compound of the formula (I), or a pharmaceutically acceptable salt thereof, as defined hereinbefore for use in the production of an anti-proliferative effect in a warm-blooded animal such as man.

According to this aspect of the invention there is provided the use of a compound of the formula (I), or a pharmaceutically acceptable salt thereof, as defined hereinbefore for use in the production of an anti-cancer effect in a warm-blooded animal such as man.

According to a further feature of the invention, there is provided the use of a compound of the formula (I), or a pharmaceutically acceptable salt thereof, as defined herein before for use in the treatment of carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma.

In a further embodiment the present invention provides the use of a compound of formula (I) or a pharmaceutically acceptable salt thereof, for the treatment or prophylaxis of disorders associated with cancer.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:

(i) antiproliferative/antineoplastic drugs and combinations thereof, as used in medical oncology, such as allylating agents (for example cis-platin, carboplatin, oxaliplatin, cyclophosphamide, nitrogen mustard, melphalan, chlorambucil, busulphan, temozolomide and nitrosoureas); antimetabolites (for example gemcitabine and antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere) polokinase inhibitors; and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function or inhibitors of SRC kinase (like 4-(6-chloro-2,3-methylenedioxyanilino)-7-[2-(4-methylpiperazin-1-yl)ethoxy]-5-tetrahydropyran-4-yloxyqyuinazoline (AZD0530; International Patent Application WO 01/94341) and N—(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661))or antibodies to Heparanase);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [Erbitux, C225]), Ras/Raf signalling inhibitors such as farnesyl transferase inhibitors(for example sorafenib (BAY 43-9006) and tipifarnib), tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), for example inhibitors of the platelet-derived growth factor family such as imatinib, and for example inhibitors of the hepatocyte growth factor family, c-kit inhibitors, abl kinase inhibitors, IGF receptor (insulin-like growth factor) kinase inhibitors and inhibitors of cell signalling through MEK, AKT and/or PI3K kinases;

(v) antiangiogenic agents such as those which inhibit the effects of vascular endothelial growth factor, (for example the anti-vascular endothelial cell growth factor antibody bevacizumab [Avastin™], and VEGF receptor tyrosine kinase inhibitors such as those disclosed in International Patent Applications WO 97/22596, WO 97/30035, WO 97/32856, WO 98/13354, 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU 11248 (sunitinib; WO 01/60814)) and compounds that work by other mechanisms (for example linomide, inhibitors of integrin αvβ3 function and angiostatin), ang 1 and 2 inhibitors;

(vi) vascular damaging agents such as Combretastatin A4 and compounds disclosed in International Patent Applications WO 99/02166, WO 00/40529, WO 00/41669, WO 01/92224, WO 02/04434 and WO 02/08213, anti bcl2;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;

x) cell cycle agents such as aurora kinase inhibitors (for example PH739358, VX-680, MLN8054, R763, MP235, MP529, VX-528, AX39459 and the specific examples mentioned in WO02/00649, WO03/055491, WO2004/058752, WO2004/058781, WO2004/058782, WO2004/094410, WO2004/105764, WO2004/113324 which are incorporated herein by reference), and cyclin dependent kinase inhibitors such as CDK2 and/or CDK4 inhibitors (for example the specific examples of WO01/14375, WO01/72717, WO02/04429, WO02/20512, WO02/66481, WO02/096887, WO03/076435, WO03/076436, WO03/076434, WO03/076433, WO04/101549 and WO04/101564 which are incorporated herein by reference); and

xi) cytotoxic agents such as gemcitibine, topoisomerase 1 inhibitors (adriamycin, etoposide) and topoisomerase II inhibitors.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In a further aspect of the present invention there is provided a compound of formula (I) or a pharmaceutically acceptable salt or an in vivo hydrolysable ester thereof in combination with simultaneous, sequential or separate dosing of an anti-tumor agent or class selected from the list herein above.

Therefore in a further embodiment the present invention provides a method for the treatment of cancer by administering to a human a compound of formula (I) or a pharmaceutically acceptable salt or an in vivo hydrolysable ester thereof in combination with simultaneous, sequential or separate dosing of an anti-tumor agent or class selected from the list herein above.

In a further aspect of the present invention there is provided the use of a compound of formula (I) or a pharmaceutically acceptable salt or an in vivo hydrolysable ester thereof in combination with simultaneous, sequential or separate dosing of an anti-tumor agent or class selected from the list herein above for use in the manufacture of a medicament for use in the treatment of cancer.

In a further aspect of the present invention there is provided the use of a compound of formula (I) or a pharmaceutically acceptable salt or an in vivo hydrolysable ester thereof in combination with simultaneous, sequential or separate dosing of an anti-tumor agent or class selected from the list herein above for use in the treatment of cancer.

The anti-cancer treatment defined herein may also include one or more of the following categories of pharmaceutical agents:

i) an agent useful in the treatment of anemia, for example, a continuous eythropoiesis receptor activator (such as epoetin alfa);

ii) an agent useful in the treatment of neutropenia, for example, a hematopoietic growth factor which regulates the production and function of neutrophils such as a human granulocyte colony stimulating factor, (G-CSF), for example filgrastim; and

iii) an anti-emetic agent to treat nausea or emesis, including acute, delayed, late-phase, and anticipatory emesis, which may result from the use of a compound of the present invention, alone or with radiation therapy, suitable examples of such anti emetic agents include neurokinin-1 receptor antagonists, 5H13 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid or Benecorten, an antidopaminergic, such as the phenothiazines (for example prochlorperazine, fluphenazine, thioridazine and mesoridazine), metoclopramide or dronabinol.

Such conjoint treatment may be achieved by way of the simultaneous, sequential or separate dosing of the individual components of the treatment. Such conjoint treatment employs the compounds of this invention within the dosage range described hereinbefore and the other pharmaceutically-active agent within its approved dosage range.

In a further aspect of the present invention there is provided a compound of formula (I) or a pharmaceutically acceptable salt or an in vivo hydrolysable ester thereof in combination with simultaneous, sequential or separate dosing of another pharmaceutical agent or class selected from the list herein above.

Therefore in a further embodiment the present invention provides a method for the treatment of cancer by administering to a human a compound of formula (I) or a pharmaceutically acceptable salt or an in vivo hydrolysable ester thereof in combination with simultaneous, sequential or separate dosing of another pharmaceutical agent or class selected from the list herein above.

In a further aspect of the present invention there is provided the use of a compound of formula (I) or a pharmaceutically acceptable salt or an in vivo hydrolysable ester thereof in combination with simultaneous, sequential or separate dosing of another pharmaceutical agent or class selected from the list herein above for use in the manufacture of a medicament for use in the treatment of cancer.

In a further aspect of the present invention there is provided the use of a compound of formula (I) or a pharmaceutically acceptable salt or an in vivo hydrolysable ester thereof in combination with simultaneous, sequential or separate dosing of another pharmaceutical agent or class selected from the list herein above for use in the treatment of cancer.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of Eg5 in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-30° C.;

(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;

(iii) in general, the course of reactions was followed by TLC or MS and reaction times are given for illustration only;

(iv) final products had satisfactory proton nuclear magnetic resonance (NMR) spectra and/or mass spectral data;

(v) yields are given for illustration only and are not necessarily those which can be obtained by diligent process development; preparations were repeated if more material was required;

(vii) when given, NMR data is in the form of delta values for major diagnostic protons, given in parts per million (ppm) relative to tetramethylsilane (TMS) as an internal standard, determined at 400 MHz using deuterated chloroform (CDCl₃) as solvent unless otherwise indicated;

(vii) chemical symbols have their usual meanings; SI units and symbols are used;

(viii) solvent ratios are given in volume:volume (v/v) terms; and

(ix) mass spectra were run with an electron energy of 70 electron volts in the chemical ionization (CI) mode using a direct exposure probe; where indicated ionization was effected by electron impact (EI), fast atom bombardment (FAB); electrospray (ESP); or atmospheric pressure chemical ionisation (APCI); values for m/z are given; generally, only ions which indicate the parent mass are reported; and unless otherwise stated, the mass ion quoted is (MH)⁺;

(x) where a synthesis is described as being analogous to that described in a previous example the amounts used are the millimolar ratio equivalents to those used in the previous example; and

(xi) the following abbreviations have been used:

THF tetrahydrofuran;

DMF N,N-dimethylformamide;

EtOAc ethyl acetate;

DCM dichloromethane; and

DMSO dimethylsulphoxide.

### Example 1

### 2,3-Dihydro-5-oxo-6-benzyl-7-{1-[N-(4-methylbenzoyl)-N-(3-aminopropyl)amino]propyl}-5H-[1,3 ]thiazolo[b 3,2-a]pyrimidine

A solution of 4N HCl in dioxane (1 ml) was added to 2,3-dihydro-5-oxo-6-benzyl-7-[1-1{-N-(4-methylbenzoyl)-N-[3-(t-butoxycarbonylamino)propyl]amino}propyl)-5H-[1,3]thiazolo[3,2-a]pyrimidine (Method 22; 103 mg, 0.18 mmol) and allowed to stir for 30 min. When deprotection was complete, the solution was concentrated in vacuo. The residue was then lyophilized from water/acetonitrile to afford 92 mg of the title compound. NMR (500 MHz, DMSO-d₆; 100° C.) 7.69 (br s, 3H) 7.25-7.32 (m, 2H) 7.09-7.22 (m, 5H) 6.87 (br s, 2H) 5.06 (br s, 1H) 4.39-4.49 (m, 2H) 3.65-3.82 (m, 4H) 3.45-3.58 (m, 2H) 2.62-2.74 (m, 2H) 2.39 (s, 3H) 1.57-1.93 (m, 4H) 0.57 (t, 3H); m/z 477 (M⁺).

### Examples 2-6

The following compounds were prepared by the procedure of Example 1.

| Ex | Compound | NMR (DMSO-d₆; 100° C.) | M/z (M⁺) | SM |
|---|---|---|---|---|
| 2 | 3,4-Dihydro-6-oxo-7- | (500MHz) 7.63(br s, 3H) 7.28(d, | 491 | Method |
| | benzyl-8-{1-[N-(4- | J=7.83Hz, 2H) 7.09-7.22(m, 5H) | | 23 |
| | methyl benzoyl)-N-(3- | 6.85(br s, 2H) 5.03(br s, 1H) | | |
| | aminopropyl)amino] | 3.98-4.11(m, 4H) 3.47-3.59(m, 2H) | | |
| | propyl}-2H,6H- | 3.20-3.28(m, 2H) 2.79-2.66(m, 2H) | | |
| | pyrimido[2,1-b] | 2.39(s, 3H) 2.22-2.31(m, 2H) | | |
| | [1,3]thiazine | 1.57-1.89(m, 4H) 0.56(t, J=7.09Hz, 3H) | | |
| 3 | 3,4-Dihydro-6-oxo-7- | 8.01(br s, 3H) 7.28-7.40(m, J=7.83Hz, | 477 | Method |
| | benzyl-8-{1-[N-(4- | 2H) 7.08-7.23(m, 5H) 7.00(d, | | 25 |
| | methyl benzyl)-N-(3- | J=6.85Hz, 2H) 3.84-4.08(m, 5H) | | |
| | aminopropyl)amino] | 3.49-3.57(m, 2H) 3.18-3.29(m, 2H) | | |
| | propyl}-2H,6H- | 3.01(br s, 2H) 2.73(s, 2H) 2.34(s, | | |
| | pyrimido[2,1-b] | 3H) 2.21-2.31(m, 2H) | | |
| | [1,3]thiazine | 1.77-2.01(m, 4H) 0.52(t, 3H) | | |
| 4 | 3,4-Dihydro-6-oxo-7- | 7.66-7.99(m, 3H) 7.25(d, J=8.80Hz, | 507 | Method |
| | benzyl-8-{1-[N-(4- | 2H) 7.08-7.20(m, 3H) 7.00(d, | | 24 |
| | methoxybenzoyl)-N-(3- | J=7.83Hz, 2H) 6.89(d, J=6.85Hz, 2H) | | |
| | aminopropyl)amino] | 5.06(s, 1H) 3.96-4.13(m, 2H) | | |
| | propyl}-2H,6H- | 3.84(s, 3H) 3.47-3.57(m, 2H) | | |
| | pyrimido[2,1-b] | 3.04-3.33(m, 6H) 2.61-2.75(m, 2H) | | |
| | [1,3]thiazine | 2.20-2.32(m, 2H) 1.57-1.94(m, 4H) | | |
| | | 0.57(t, J=7.34Hz, 3H) | | |
| 5 | 5-Oxo-6-benzyl-7-{1- | (500MHz) 8.03(d, J=4.89Hz, 1H) | 493 | Method |
| | [N-(3-fluoro-4- | 7.58(d, J=4.89Hz, 1H) 7.43-7.56(m, | | 26 |
| | methylbenzoyl)-N-(3- | 3H) 7.31-7.39(m, 2H) | | |
| | aminopropyl)amino] | 7.10-7.23(m, 3H) 7.02(m, 4H) 5.24(br s, 1H) | | |
| | propyl}-5H- | 4.03(d, J=15.16Hz, 2H) 3.53(s, 2H) | | |
| | [1,3]thiazolo[3,2- | 2.65(s, 2H) 2.31(s, 3H) | | |
| | a]pyrimidine | 1.97-2.08(m, 1H) 1.82-1.95(m, 1H) 1.80(m, 1H) | | |
| | | 1.59(m, 1H) 0.56-0.67(m, 3H) | | |
| 6 | 2-{1-[N-(Naphth-2- | (500MHz) 8.30(br s, 1H) | 496 | Method |
| | ylcarbonyl)-N-(2-amino | 8.00-7.80(m, 7H) 7.70(m, 2H) 7.20(t, 1H) | | 32 |
| | ethyl)amino]propyl}-3- | 6.78(t, 1H), 4.10(m, 2H) | | |
| | benzyl-4-oxo-6,7,8,9- | 3.70-3.55(m, 5H), 2.77(br m, 4H), | | |
| | tetrahydro-4H- | 1.37-1.22(m, 6H) 1.00-0.90(m, 3H) | | |
| | pyrido[1,2-a]pyrimidine | | | |

### Example 7

### 2-{1-[N-(4-Chlorobenzoyl)-N-(2-dimethylaminoethyl)amino]propyl}-3-benzyl-4-oxo-6,7,8,9-tetrahydro-4H-pyrido[1,2-a]pyrimidine

To a solution of 3-benzyl-2-[1-(2-dimethylamino-ethylamino)-propyl]-6,7,8,9-tetrahydro-pyrido[1,2-a]pyrimidin-4-one (Method 30; 2 mg, 5.43 μmol) in DCM (1 ml), was added an aqueous solution of potassium carbonate (10 mg/1 ml) and 4-chloro benzoyl chloride (1.2 eq., 6.5 μmol, 1 μL). When the reaction was complete, the layers were separated and dried over Mg₂SO₄, filtered, and concentrated in vacuo. The compound was purified by reverse phase chromatography (0.1% TFA/water-0.1% TFA/CH₃CN) to afford 3 mg (91% isolated yield) of the title compound. NMR (300 MHz, MeOD) 7.75 (m, 2H) 7.40-6.90 (m, 7H), 4.65-4.50 (m, 2H), 4.40-4.30 (m, 1H), 3.75 (m, 2H) 3.60-3.35 (m, 4H), 3.00 (hidden by H₂O peak, 2H), 3.00-2.85 (m, 6H), 2.30 (s, 6H), 1.30 (m, 3H); m/z 508 (M⁺).

Preparation of Starting Materials

Method 1

### Ethyl 4,4-dimethoxy-3-oxobutanoate

To a solution of dimethoxy methyl acetate (25 g, 0.19 mol) in EtOAc (75 ml), was added sodium hydride (8.5 g, 0.21 mol) in portions. After all the sodium hydride was added, the mixture was set to reflux for 10 hours. The reaction mixture was poured onto cold water followed by pH adjustment to 3-4 using 3N HCl solution. The layers were separated and the organic layer was dried, filtered, and concentrated in vacuo to afford 34 g (96% crude recovery) of the title compound, which was used further without purification.

Method 2

### Ethyl 2-benzyl-4,4-dimethoxy-3-oxobutanoate

A solution of ethyl 4,4-dimethoxy-3-oxobutanoate (Method 1; 20 g, 0.105 mol), benzyl chloride (12.2 ml, 0.106 mol), and sodium ethoxide (7 g, 0.105 mol) in ethanol was set to reflux for 10 hours. The reaction mixture was then cooled down to room temperature and concentrated in vacuo. The residue was then diluted in EtOAc and partitioned with water. The layers were separated and the organic layer was dried, filtered, and concentrated in vacuo to afford 24.2 g (82% crude recovery) of the title compound, which was used further without purification.

Method 3

### 5-Benzyl-6-(dimethoxymethyl)-2-thioxo-2,3-dihydropyrimidin-4(1H)-one

To a solution of ethyl 2-benzyl-4,4-dimethoxy-3-oxobutanoate 3 (Method 2; 10 g, 35.7 mmol) in ethanol (100 ml), was added thiourea (2.58 g, 33.9 mmol) followed by sodium ethoxide (2.43 g, 35.7 mmol). The reaction mixture was set to reflux for 5 h. When the reaction was complete, the mixture was poured onto cold water and pH adjusted to 3 using 3N HCl solution. The solution was then partitioned with EtOAc and the layers were separated. The organic layer was dried, filtered, and concentrated in vacuo. The residue was triturated from hexanes to afford 4.16 g (42% isolated yield) of the title compound. NMR (300 MHz) 7.10-7.39 (m, 5H), 5.26 (s, 1H), 3.82 (s, 2H), 3.31 (s, 6H); m/z 293 (M⁺).

Method 4

### 6-Benzyl-7-(dimethoxymethyl)-2,3-dihydro-5H-[1,3]thiazolo[3,2-a]pyrimidin-5-one

To a solution of 5-benzyl-6-(dimethoxymethyl)-2-thioxo-2,3-dihydropyrimidin-4(1H)-one (Method 3; 2.14 g, 7.33 mmol) in DMF (20 ml), was added K₂CO₃ (5.5 g, 40 mmol) and 1,2-dibromoethane (0.76 ml, 8.79 mmol). The mixture was set to heat to 85° C. for 10 hours. The reaction mixture was quenched by the addition of water and partitioned with EtOAc. The layers were separated and the organic layer was dried, filtered, and concentrated in vacuo. The residue was purified on silica gel using 80-90% EtOAc/hexanes to afford 0.91 g (39% isolated yield) of the title compound and 0.83 g (35% isolated yield) of 6-benzyl-5-(dimethoxymethyl)-2,3-dihydro-7H-[1,3]thiazolo[3,2-a]pyrimidin-7-one 6. NMR (300 MHz) 7.07-7.45 (m, 5H) 5.22-5.34 (m, 1H) 4.41-4.55 (m, J=7.72, 7.72 Hz, 2H) 4.00 (s, 2H) 3.36-3.53 (m, 8H); m/z 319 (M⁺)

Methods 5-6

The following compounds were prepared by the procedure of Method 4 using the allylating agent shown.

| Method | Compound Name | m/z | Alkylating agent |
|---|---|---|---|
| 5 | 7-Benzyl-8-dimethoxymethyl- | 333 | 1,3-dibromopropane |
| | 3,4-dihydro-2H-pyrimido[2,1- | | |
| | b][1,3]thiazin-6-one | | |
| 6 | 6-Benzyl-7-dimethoxymethyl- | 317 | 1,2-dibromoethene |
| | thiazolo[3,2-a]pyrimidin-5-one | | |

Method 7

### 6-Benzyl-5-oxo-2,3-dihydro-5H-[1,3]thiazolo[3,2-a]pyrimidine-7-carbaldehyde

A mixture 6-benzyl-7-(dimethoxymethyl)-2,3-dihydro-5H-[1,3]thiazolo[3,2-a]pyrimidin-5-one (Method 4; 1.5 g) in 30 ml of aqueous sulfuric acid solution (10% v/v) was heated at 80° C. for 15-30 min. The reaction was monitored by consumption of starting material. When complete, the reaction mixture was partitioned with DCM and layers were separated. The organic layer was dried, filtered, and concentrated in vacuo to afford 1.17 g (91% recovered yield) of the title compound. This compound was used further without purification. NMR (300 MHz) 9.96 (s, 1H) 7.02-7.37 (m, 5H) 4.33-4.48 (m, 2H) 4.19(s, 2H) 3.37-3.46 (m, 2H).

Methods 8-9

The following compounds were prepared by the procedure of Method 7 using the starting material indicated.

| Method | Compound Name | NMR | SM |
|---|---|---|---|
| 8 | 7-Benzyl-6-oxo-3,4-dihydro-2H,6H- | (300MHz) 9.90(s, 1H) | Method 5 |
| | pyrimido[2,1-b][1,3]thiazine-8- | 7.23-7.35(m, 2H) 7.05-7.18(m, 3H) | |
| | carbaldehyde | 4.19(s, 2H) 3.98-4.10(m, | |
| | | 2H) 3.04-3.22(m,2H) | |
| | | 2.13-2.29(m, 2H). | |
| 9 | 6-Benzyl-5-oxo-5H-thiazolo[3,2- | 10.08(s, 1H) 7.94(d, J=4.89Hz, | Method 6 |
| | a]pyrimidine-7-carbaldehyde | 1H) 7.31(d, J=6.85Hz, 2H) | |
| | | 7.07-7.27(m, 3H) 7.05(d, | |
| | | J=4.89Hz, 1H) 4.35(s, 2H) | |
| | | 2.90(t, J=7.83Hz, 2H) 2.63(t, | |
| | | J=7.83Hz, 2H). | |

Method 10

### 6-Benzyl-7-(1-hydroxypropyl)-2,3-dihydro-5H-[1,3]thiazolo[3 2-a]pyrimidin-5-one

To a cold solution of 6-benzyl-5-oxo-2,3-dihydro-5H-[1,3]thiazolo[3,2-a]pyrimidine-7-carbaldehyde (Method 7; 1.17 g, 4.3 mmol) in tetrahydrofuran (15 ml) at −40° C., was added ethyl magnesium bromide (1M in THF; 5.1 ml, 5.1 mmol). The reaction mixture was allowed to stir at −40° C. for a few hours before quenching with 1M NH₄Cl solution. The reaction mixture was partitioned with EtOAc and the layers were separated. The organic layer was dried, filtered, and concentrated in vacuo to afford 450 mg (33% isolated yield) of the title compound after silica gel purification (10% EtOAc/DCM).NMR 7.06-7.24 (m, 5H) 4.55 (s, 1H) 4.40 (t, J=7.34 Hz, 2H) 3.77 (d, J=5.87 Hz, 2H) 3.39 (t, J=7.83 Hz, 2H) 3.04 (d, J=8.80 Hz, 1H) 1.31-1.60 (m, 2H) 0.86 (t, J=7.34 Hz, 3H); m/z 303 (M⁺).

Methods 11-12

The following compounds were prepared by the procedure of Method 10 using the starting material indicated.

| Method | Compound Name | m/z | SM |
|---|---|---|---|
| 11 | 7-Benzyl-8-(1-hydroxy-propyl)-3,4-dihydro-2H | 317 | Method |
| | pyrimido[2,1-b][1,3]thiazin-6-one | | 8 |
| 12 | 6-Benzyl-7-(1-hydroxy-propyl)-thiazolo[3,2- | 301 | Method |
| | a]pyrimidin-5-one | | 9 |

Method 13

### 2-[1-(6-benzyl-5-oxo-2,3-dihydro-5H-[1,3]thiazolo[3,2-a]pyrimidin-7-yl)propyl]-1H-isoindole-1,3(2H)-dione

To a solution of 6-benzyl-7-(1-hydroxypropyl)-2,3-dihydro-5H-[1,3]thiazolo[3,2-a]pyrimidin-5-one (Method 10; 413 mg, 1.37 mmol) in THF (13.7 ml), was added phthalimide (221 mg, 1.5 mmol) and triphenylphosphine (393 mg, 1.5 mmol). As the solution was cooled to 0° C., diisopropyl azodicarboxylate (DIAD) (0.3 ml, 1.5 mmol) was added and the solution was allowed to stir at cold temperature for 30 min. The cooling bath was removed and the reaction was allowed to reach ambient temperature. When the reaction was complete, the mixture was quenched with water and partitioned with EtOAc. The layers were separated, and the organic layer was dried, filtered, and concentrated in vacuo to afford 450 mg of the title compound after silica gel purification (1:1 hexanes/EtOAc). M/z 432 (M⁺).

Methods 14-15

The following compounds were prepared by the procedure of Method 13 using the starting material indicated.

| Method | Compound Name | m/z | SM |
|---|---|---|---|
| 14 | 2-[1-(7-Benzyl-6-oxo-3,4-dihydro-2H,6H- | 446 | Method 11 |
| | pyrimido[2,1-b][1,3]thiazin- | | |
| | 8-yl)-propyl]-isoindole-1,3-dione | | |
| 15 | 2-[1-(6-Benzyl-5-oxo-5H-thiazolo[3,2- | 430 | Method 12 |
| | a]pyrimidin-7-yl)-propyl]-isoindole- | | |
| | 1,3-dione | | |

Method 16

### 7-(1-Aminopropyl)-6-benzyl-2,3-dihydro-5H-[1,3]thiazolo[3,2-a]pyrimidin-5-one

A solution of 2-[1-(6-benzyl-5-oxo-2,3-dihydro-5H-[1,3]thiazolo[3,2-a]pyrimidin-7-yl)propyl]-1H-isoindole-1,3(2H)-dione (Method 13; 450 mg) in ethanol (10 ml) was treated with hydrazine hydrate (0.2 ml, 4.18 mmol) and left to stir overnight. The resulting mixture was filtered through a pad of diatomaceous earth followed by several washes of ethanol. The filtrate was concentrated in vacuo to afford 256 mg (62% overall yield for the 2 steps) of the title compound, which was used further without purification. M/z 302 (M⁺).

Methods 17-18

The following compounds were prepared by the procedure of Method 16 using the starting material indicated.

| Method | Compound Name | m/z | SM |
|---|---|---|---|
| 17 | 8-(1-Amino-propyl)-7-benzyl-3,4-dihydro-2H- | 316 | Method |
| | pyrimido[2,1-b][1,3]thiazin-6-one | | 14 |
| 18 | 7-(1-Amino-propyl)-6-benzyl-thiazolo[3,2- | 300 | Method |
| | a]pyrimidin-5-one | | 15 |

Method 19

### tert-Butyl (3-{[1-(6-benzyl-5-oxo-2,3-dihydro-5H-[1,3]thiazolo[3,2-a]pyrimidin-7-yl)propyl]amino}propyl carbamate

To a mixture of 7-(1-aminopropyl)-6-benzyl-2,3-dihydro-5H-[1,3]thiazolo[3,2-a]pyrimidin-5-one (Method 16; 256 mg, 0.85 mmol) and tert-butyl (3-oxopropyl)carbamate (162 mg, 0.93 mmol) in dichloroethane (4 ml), was added Na(OAc)₃BH (198 mg, 0.93 mmol). The resulting mixture was allowed to stir at ambient temperature overnight. The reaction mixture was quenched with water and saturated sodium hydrogencarbonate solution, and partitioned with DCM. The layers were separated and the organic layer was dried, filtered, and concentrated in vacuo. The residue was purified on silica gel (5% MeOH/DCM) to afford 150 mg (38% isolated yield) of the title compound. M/z 459 (M⁺).

Methods 20-21

The following compounds were prepared by the procedure of Method 19 using the starting material indicated.

| Method | Compound Name | m/z | SM |
|---|---|---|---|
| 20 | {3-[1-(7-benzyl-6-oxo-3,4-dihydro-2H,6H- | 473 | Method 17 |
| | pyrimido[2,1-b][1,3]thiazin-8-yl)- | | |
| | propylamino]-propyl}-carbamic | | |
| | acid tert-butyl ester | | |
| 21 | {3-[1-(6-benzyl-5-oxo-5H thiazolo[3,2- | 457 | Method 18 |
| | a]pyrimidin-7-yl)-propylamino]-propyl}- | | |
| | carbamic acid tert-butyl ester | | |

Method 22

### 2,3-Dihydro-5-oxo-6-benzyl-7-[1-{N-(4-methylbenzoyl)-N-[3-(t-butoxycarbonylamino)propyl]amino}propyl)-5H-[1,3]thiazolo[3,2-a]pyrimidine

To a solution of tert-butyl (3-{[1-(6-benzyl-5-oxo-2,3-dihydro-5H-[1,3]thiazolo[3,2-a]pyrimidin-7-yl)propyl]amino}propyl)carbamate (Method 19; 142 mg, 0.31 mmol) in DCM (1.6 ml), was added triethylamine (0.047 ml, 0.34 mmol) and 4-methyl benzoyl chloride (0.045 ml, 0.34 mmol). When the reaction was complete, the mixture was concentrated in vacuo and purified on silica gel (20% EtOAc/DCM) to afford 103 mg (58% isolated yield) of the title compound. M/z 577 (M⁺).

Methods 23-26

The following compounds were prepared by the procedure of Method 22 using the starting materials and acylating/alkylating agent indicated indicated.

| | | | | Acylating/ |
|---|---|---|---|---|
| Method | Compound Name | m/z | SM | alkylating agent |
| 23 | 3,4-Dihydro-6-oxo-7-benzyl-8-(1-{N-(4- | 591 | Method 20 | 4-methyl-benzoyl |
| | methylbenzoyl)-N-[3-(t-butoxy | | | chloride |
| | carbonylamino)propyl]amino}propyl)- | | | |
| | 2H,6H-pyrimido[2,1-b][1,3]thiazine | | | |
| 24 | 3,4-Dihydro-6-oxo-7-benzyl-8-(1-{N-(4- | 607 | Method 20 | 4-methoxy- |
| | methoxybenzoyl)-N-[3-(t-butoxy | | | benzoyl chloride |
| | carbonylamino)propyl]amino}propyl)- | | | |
| | 2H,6H-pyrimido[2,1-b][1,3]thiazine | | | |
| 25 | 3,4-Dihydro-6-oxo-7-benzyl-8-(1-{N-(4- | 577 | Method 20 | 4-methyl benzyl |
| | methylbenzyl)-N-[3-(t-butoxy | | | chloride |
| | carbonylamino)propyl]amino}propyl)- | | | |
| | 2H,6H-pyrimido[2,1-b][1,3]thiazine | | | |
| 26 | 5-oxo-6-benzyl-7-(1-{N-(3-fluoro-4- | 593 | Method 21 | 3-fluoro, 4- |
| | methylbenzoyl)-N-[3-(t-butoxy | | | methyl-benzoyl |
| | carbonylamino)propyl]amino}propyl)- | | | chloride |
| | 5H-[1,3]thiazolo[3,2-a]pyrimidine | | | |

Method 27

### 3-Benzyl-2-dimethoxymethyl-6,7,8,9-tetrahydro-pyrido[1,2-a]pyrimidin-4-one

To a solution of ethyl 2-benzyl-4,4-dimethoxy-3-oxobutanoate (Method 2; 5 g, 17.85 mmol) in ethanol (50 ml), was added 2-iminopiperidine hydrochloride (1.5 eq., 3.60 g, 26.7 mmol) followed by sodium ethoxide (1.21 g, 17.85 mmol). The reaction mixture was reacted in the microwave at 120° C. for 1 hour. When the reaction was complete, the solution was evaporated to dryness and redissolved in EtOAc. The compound was purified by flash chromatography (6% MeOH/EtOAc) to yield only the title compound (4.03 g, 12.8 mmol) in 72% yield. NMR (300 MHz) 7.35-7.15 (m, 5H) 5.40 (s, 1H) 4.10 (s, 2H) 4.00-3.90 (t, 2H), 3.40 (s, 6H), 3.20 (t, 2H) 2.10-1.90 (m, 4H); m/z 315 (M⁺).

Method 28

### 3-Benzyl-4-oxo-6,7,8,9-tetrahydro-4H-pyrido[1,2-a]pyrimidine-2-carbaldehyde

A mixture of 3-benzyl-2-dimethoxymethyl-6,7,8,9-tetrahydro-pyrido[1,2-a]pyrimidin-4-one (Method 27; 0.40 g, 1.27 mmol) in 30 ml of aqueous sulfuric acid solution (10% v/v, 50 ml) was heated at 80° C. for 50 min. The reaction was monitored by consumption of starting material. When complete, the reaction mixture was partitioned with DCM and layers were separated. The organic layer was dried over Mg₂SO₄, filtered, and concentrated in vacuo to afford 0.302 g (89% recovered yield) of the title compound. This compound was used further without purification. M/z 269 (M⁺).

Method 29

### 3-Benzyl-2-(1′-hydroxy-propyl)-6,7,8,9-tetrahydro-pyrido[1,2-a]pyrimidin-4-one

To a cold solution of 3-benzyl-4-oxo-6,7,8,9-tetrahydro-4H-pyrido[1,2-a]pyrimidine-2-carbaldehyde (Method 28; 0.302 g, 1.13 mmol) in THF (30 ml) at −78° C., was added ethyl magnesium bromide (1M in THF; 1.48 ml, 1.48 mmol). The reaction mixture was allowed to stir at −78° C. for 1 hour before quenching with 1M NH₄Cl solution. The reaction mixture was partitioned with EtOAc and the layers were separated. The organic layer was dried over Mg₂SO₄, filtered, and concentrated in vacuo to afford 264 mg (68% isolated yield from two steps) of the title compound after silica gel purification (4% MeOH/EtOAc). M/z 299 (M⁺).

Method 30

### 3-Benzyl-2-[1-(2-dimethylamino-ethylamino)-propyl]-6,7,8,9-tetrahydro-pyrido[1,2-a]pyrimidin-4-one

3-Benzyl-2-(1-hydroxy-propyl)-6,7,8,9-tetrahydro-pyrido[1,2-a]pyrimidin-4-one (Method 29; 40 mg, 0.134 mmol) was dissolved in anhydrous DCM (3.5 ml) and to this was added 2,6-lutidine (2 eq., 0.268 mmol, 31 μl) and trifluoroacetic anhydride (1.2 eq., 0.161 mmol, 23 μl). The resulting mixture was reacted under microwave conditions at 110° C. for 30 mins. The reaction mixture was evaporated to dryness and redissolved in anhydrous DCM (3 ml). To this was added N*1*,N*1*-dimethyl-ethane-1,2-diamine (1.2 eq., 0.161 mmol, 1.3 mg) and diisopropylethylamine (1.2 eq., 0.161 mmol, 28 μl). The resulting mixture was reacted under microwave conditions at 80° C. for 30 mins. The reaction mixture was evaporated to dryness and the residue was purified on silica gel (10% MeOH/EtOAc) to afford 2 mg (4% isolated yield) of the title compound. M/z 369 (M⁺).

Method 31

The following compound was prepared by the procedure of Method 30 using 3-benzyl-2-(1-hydroxy-propyl)-6,7,8,9-tetrahydro-pyrido[1,2-a]pyrimidin-4-one (Method 29) and the alkylating agent indicated.

| Method | Compound Name | m/z | Alkylating agent |
|---|---|---|---|
| 31 | {2-[1-(3-Benzyl-4-oxo-6,7,8,9- | 441 | (2-Amino-ethyl)- |
| | tetrahydro-4H-pyrido[1,2-a] | (M⁺) | carbamic acid tert- |
| | pyrimidin-2-yl)-propylamino] | | butyl ester |
| | -ethyl}-carbamic acid tert- | | |
| | butyl ester | | |

Method 32

The following compound was prepared by the procedure of Example 7 using {2-[1-(3-benzyl-4-oxo-6,7,8,9-tetrahydro-4H-pyrido[1,2-a]pyrimidin-2-yl)-propylamino]-ethyl}-carbamic acid tert-butyl ester (Method 31) the acylating agent indicated.

| Method | Compound Name | m/z | Acylating agent |
|---|---|---|---|
| 32 | {2-[[1-(3-Benzyl-4-oxo-6,7,8,9- | 596 | 2-napthoyl |
| | tetrahydro-4H-pyrido[1,2-a] | (M⁺) | chloride |
| | pyrimidin-2-yl)-propyl]-(naphthalene-2- | | |
| | carbonyl)-amino]-ethyl}-carbamic | | |
| | acid tert-butyl ester | | |

1. A compound of formula (I): 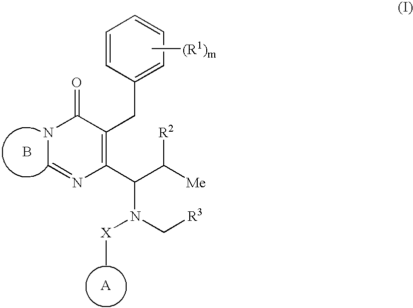
wherein:
R¹ is fluoro;
m is 0-5;
R² is hydrogen or methyl;
R³ is a carbon linked —NR⁴— containing heterocyclic ring or R³ is C₁₋₃alkyl substituted by —NR⁵R⁶; wherein R³ may be optionally substituted on carbon by one or more R⁷;
X is —C(O)— or —CH₂—;
Ring A is a carbocyclyl or heterocyclyl; wherein Ring A may be optionally substituted on carbon by one or more R⁸; and wherein if said heterocyclyl contains an additional NH that nitrogen may be optionally substituted by R⁹;
Ring B is fused to the pyrimidone ring of formula (I) as shown and is a 5 or 6 membered fused carbocyclic ring or 5 or 6 membered fused heterocyclic ring; wherein Ring B may be optionally substituted on carbon by one or more R¹⁰; and wherein if said 5 or 6 membered fused heterocyclic ring contains an additional NH that nitrogen may be optionally substituted by R¹¹;
R⁴ is selected from hydrogen, C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R⁵ and R⁶ are independently hydrogen or C₁₋₆alkyl; or R⁵ and R⁶ together with the nitrogen to which they are attached form a nitrogen containing heterocycle; wherein said C₁₋₆alkyl or said nitrogen containing heterocycle may be independently optionally substituted on carbon by one or more R¹²; and wherein if said nitrogen containing heterocycle contains an additional NH that nitrogen may be optionally substituted by R¹³;
R⁸, R¹⁰ and R¹² are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino; wherein R⁸, R¹⁰ and R¹² may be independently optionally substituted by R¹⁴;
R⁹, R¹¹ and R¹³ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R⁷ and R¹⁴ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof.

2. A compound of formula (I), or a pharmaceutically acceptable salt thereof, according to claim 1 wherein m is 0.

3. A compound of formula (I), or a pharmaceutically acceptable salt thereof, according to claim 1 wherein R² is hydrogen.

4. A compound of formula (I), or a pharmaceutically acceptable salt thereof, according to claim 1 wherein R³ is C₁₋₃alkyl substituted by —NR⁵R⁶.

5. A compound of formula (I), or a pharmaceutically acceptable salt thereof, according to claim 1 wherein X is —C(O)—.

6. A compound of formula (I), or a pharmaceutically acceptable salt thereof, according to claim 1 wherein X is —CH₂—.

7. A compound of formula (I), or a pharmaceutically acceptable salt thereof, according to claim 1 wherein Ring A is a carbocyclyl; wherein Ring A may be optionally substituted on carbon by one or more R⁸; wherein R⁸ is halo, C₁₋₆alkyl or C₁₋₆alkoxy.

8. A compound of formula (I), or a pharmaceutically acceptable salt thereof, according to claim 1 wherein Ring B is a 5 or 6 membered fused carbocyclic ring or a 5 or 6 membered fused heterocyclic ring.

9. A compound of formula (I): 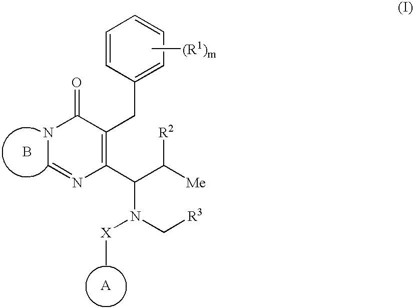
wherein:
m is 0;
R² is hydrogen;
R³ is methyl or ethyl substituted by —NR⁵R⁶;
X is —C(O)— or —CH₂—;
Ring A is 4-methyphenyl, 4-methoxyphenyl, 3-fluoro-4-methylphenyl, naphth-2-yl or 4-chlorophenyl;
Ring B and the pyrimidone to which it is attached form 2,3-dihydro-5-oxo-5H-[1,3]thiazolo[3,2-a]pyrimidine, 3,4-dihydro-6-oxo-2H,6H-pyrimido[2,1-b][1,3]thiazine, 5-oxo-5H-[1,3]thiazolo[3,2-a]pyrimidine or 4-oxo-6,7,8,9-tetrahydro-4H-pyrido[1,2-a]pyrimidin-2-yl;
R⁵ and R⁶ are independently hydrogen or methyl; or a pharmaceutically acceptable salt thereof.

10. A compound of formula (I): 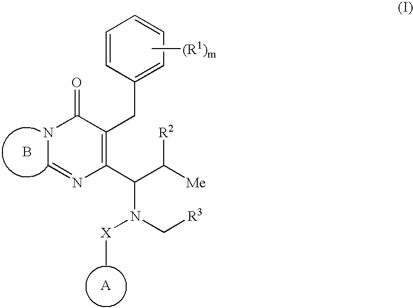
selected from:
2,3-dihydro-5-oxo-6-benzyl-7-{1-[N-(4-methylbenzoyl)-N-(3-aminopropyl)amino]propyl}-5H-[1,3]thiazolo[3,2-a]pyrimidine;
3,4-dihydro-6-oxo-7-benzyl-8-{1-[N-(4-methyl benzoyl)-N-(3-amino propyl)amino]propyl}-2H,6H-pyrimido[2,1-b][1,3]thiazine;
3,4-dihydro-6-oxo-7-benzyl-8-{1-[N-(4-methyl benzyl)-N-(3-amino propyl)amino]propyl}-2H,6H-pyrimido[2,1-b][1,3]thiazine;
3,4-dihydro-6-oxo-7-benzyl-8-{1-[N-(4-methoxybenzoyl)-N-(3-aminopropyl)amino]propyl}-2H,6H-pyrimido[2,1-b][1,3]thiazine;
5-oxo-6-benzyl-7-{1-[N-(3-fluoro-4-methyl benzoyl)-N-(3-aminopropyl)amino]propyl}-5H-[1,3]thiazolo[3,2-a]pyrimidine;
2-{1-[N-(naphth-2-ylcarbonyl)-N-(2-amino ethyl)amino]propyl}-3-benzyl-4-oxo-6,7,8,9-tetrahydro-4H-pyrido[1,2-a]pyrimidine; and
2-{1-[N-(4-chlorobenzoyl)-N-(2-dimethylaminoethyl)amino]propyl}-3-benzyl-4-oxo-6,7,8,9-tetrahydro-4H-pyrido[1,2-a]pyrimidine;
or a pharmaceutically acceptable salt thereof.

11. A process for preparing a compound of formula (I) or a pharmaceutically acceptable salt thereof, as claimed in claim 1 which process, wherein variable groups are, unless otherwise specified, as defined in claim 1, comprises of:
Process a) when X is —C(O)—; reacting a quinazolinone of the formula (II) 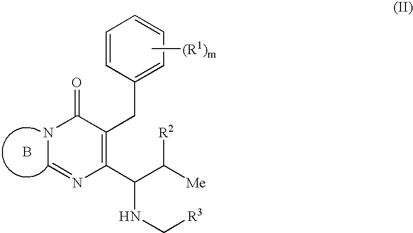
with an acid of formula (III): 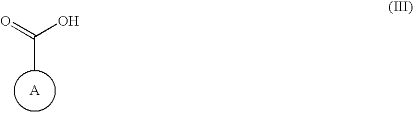
or an activated acid derivative thereof;
Process b) where X is —CH₂—; reacting a compound of the formula (II) with a compound of formula (V): 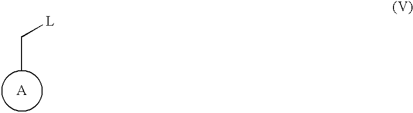
wherein L is a displaceable group;
Process c) for compounds of formula (I) wherein R³ is C₁₋₃alkyl substituted by —NR⁵R⁶ and optionally substituted on carbon by one or more R⁷; reacting a compound of formula (VI): 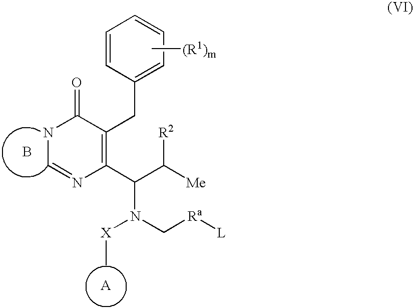
wherein R^{a} is C₁₋₃alkylene optionally substituted on carbon by one or more R⁷; and wherein L is a displaceable group; with an compound of formula (VII):
HNR⁵R⁶ (VII)
Process d) reaction of an amine of formula (VIII): 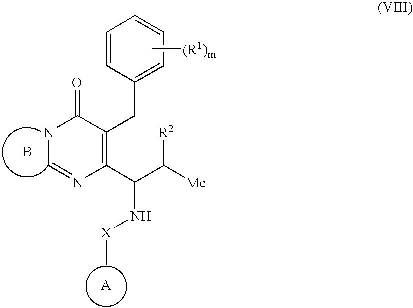
with a compound of formula (IX) 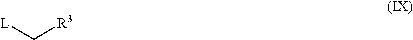
wherein L is a displaceable group;
and thereafter if necessary:
i) converting a compound of the formula (I) into another compound of the formula (I);
ii) removing any protecting groups;
iii) forming a pharmaceutically acceptable salt.

12. A pharmaceutical composition which comprises a compound of the formula (I), or a pharmaceutically acceptable salt thereof, as claimed in claim 1, in association with a pharmaceutically-acceptable diluent or carrier.

13-16. (canceled)

17. A method for producing a Eg5 inhibitory effect in a warm-blooded animal, such as man, in need of such treatment which comprises administering to said animal an effective amount of a compound of formula (I), or a pharmaceutically acceptable salt thereof, as claimed in claim 1.

18. A method for producing an anti-cancer effect in a warm-blooded animal, such as man, in need of such treatment which comprises administering to said animal an effective amount of a compound of formula (I), or a pharmaceutically acceptable salt thereof, as claimed in claim 1.

19. A method of treating carcinomas of the brain, breast, ovary, lung, colon and prostate, multiple myeloma leukemias, lymphomas, tumors of the central and peripheral nervous system, melanoma, fibrosarcoma, Ewing's sarcoma and osteosarcoma, in a warm-blooded animal, such as man, in need of such treatment which comprises administering to said animal an effective amount of a compound of formula (I) or a pharmaceutically acceptable salt thereof as claimed in claim 1.

20-22. (canceled)
